(19) United States
(12) Patent Application Publication
(10) Pub. No.: US 2015/0238772 A1
(43) Pub. Date: Aug. 27, 2015

(54) NONINVASIVE PHOTO-ULTRASONIC BIOSTIMULATION DEVICE AND METHOD OF USE

(71) Applicants: Joseph J. Meyer, St. Petersburg, FL (US); Edmond P. Wong, Kaneohe, HI (US)

(72) Inventors: Joseph J. Meyer, St. Petersburg, FL (US); Edmond P. Wong, Kaneohe, HI (US)

(21) Appl. No.: 14/187,992

(22) Filed: Feb. 24, 2014

(51) Int. Cl. A61N 5/06; A61N 7/00

(52) U.S. Cl. CPC A61N 5/06; A61N 7/00

(57) ABSTRACT

A photo-ultrasonic biostimulation device and method of use for treating internal organ dysfunction and failure by restoring homeostasis and reversing generalized cell, tissue, and organ inflammation and edema throughout the VAST and thereby supporting the healthy functional automaticity of the body. This is achieved by the controlled exposure of the mitochondria, cells, tissues, and organ in the VAST, including the organs and tissues exhibiting dysfunction and the interdependent organs, with the precise dosimetry of the synergetic combination of specific wavelengths and tolerances of electromagnetic and ultrasonic energy. Such medical device is to be used concurrently with standard best medical practices of diagnosis and treatment and is designed to supplement and significantly improve outcomes.

### FIELD OF THE PRESENT DISCLOSURE

This disclosure relates generally to noninvasive or minimally invasive medical devices and methods of use, and more particularly to a medical device and system designed to treat (intervene, impede, arrest, and frequently reverse) organ dysfunction and organ failure by reestablishing internal homeostasis both in affected organs and throughout the body using metered photo and ultrasonic biostimulation. Such medical device is to be used concurrently with standard best medical practices of diagnosis and treatment and is designed to supplement and significantly improve outcomes.

### BACKGROUND OF THE RELATED ART

Electromagnetic energy in the wavelength range of infrared to visible light have been used to treat a wide range of medical conditions such as pain, inflammation, various skin conditions, stimulation of healing superficial wounds and nerve damage, and the regeneration of hair. Therapy of this variety is often called photo biostimulation or low-level light therapy (“LLLT”). The majority of the conditions that have traditionally been treated with photo biostimulation or LLLT therapy are relatively superficial; however, more recently research has shown benefits may be achieved in deeper tissue by treating such tissue with metered exposure to specific wavelengths of electromagnetic waves capable of deeper penetration.

In order for biostimulation to occur, the electromagnetic energy must be absorbed at the cellular level by the intended target organ or tissue. When targeting internal organs or tissue that exist physically deeper in the body, electromagnetic energy similar in the wavelength spectrum to near infrared/infrared are more effective because unlike the green, blue, or violet wavelength ranges, the near infrared/infrared wavelengths penetrate deeper through superficial tissue. Other spectrum ranges may reach the deeper tissue as well but may not be absorbed by the target tissue in a manner that provides for effective photo biostimulation.

When electromagnetic energy is absorbed by a molecule within a cell of the targeted organ or tissue, the electrons of that molecule are temporarily raised into a higher energy state. Subsequently, the molecule's electrons typically return to their former energy state transferring the absorbed energy in some manner. Examples of the manner through which this transfer may occur include: re-emitting electromagnetic energy; expelling the energy as heat; and/or expending the energy through a photochemical reaction. The third exemplar is believed to be the most relevant in regards to photo biostimulation therapy.

While many of the precise mechanisms are not yet completely understood, it is widely accepted that some of the most beneficial photochemical reactions that involve the mitochondria and assists the mitochondria in the production of adenine triphosphate (“ATP”). It is has been demonstrated that when the outer membrane of the mitochondria is exposed to electromagnetic energy in the wavelength range of 600 to 1000 nm that the photoreceptor, cytochrome c oxidase, absorbs the energy and, through a series of photochemical reactions, stimulates the mitochondria to accelerate ATP production. Accelerated ATP production, in turn, leads to an overall improved cellular metabolic state and function including accelerated rate of cellular mitosis and improved protein synthesis with the result of improving tissue healing function. This near infrared/infrared photon bombardment of the outer layer of the mitochondria also increases nitric oxide availability, which critically promotes both microvascular and microlymphatic dilatation with the net result of improving toward optimal the quality of the extracellular fluid (the universal internal environment which bathes all cells and must be at or near optimal to environmentally support cell, tissue, and organ health and function). This, along with increased ATP production, is absolutely essential to reestablishing homeostasis in cells, tissues, and organs since improved extracellular fluid provides improved concentration and availability of essential cellular inputs of O2, glucose, and molecular nutrients, molecular messengers (hormonal), and factors (enzymatic and ionic). It is further theorized that the overall improved cellular state and function established through increased ATP production may lead to additional benefits such as improved mitochondrial retrograde nuclear signaling influencing both epigenetic nuclear and mitochondrial gene up regulation, increased mitochondrial biogenesis, increased protein synthesis, increased rate of mitosis, and normalized mitochondrial apoptosis (all of which produce a cascade of positive cellular and tissue actions, which thereby promote healing and recovery).

Currently, photo biostimulation is not being widely used to treat internal organ dysfunction and/or failure but its use is enjoying increased acceptance and has a promising future because of its noninvasive or minimally invasive nature and because it promotes the body's own natural healing processes. Photo biostimulation is likely to gain further currency as researchers discover and develop methods to increase its efficiency and effectiveness.

One method in which the effectiveness of photo biostimulation can be immediately increased involves the scope of the treatment approach. Often times, when an internal organ or tissue is exhibiting signs of dysfunction or failure and the selected therapy is photo biostimulation, only the identified dysfunctional or failing organ or tissue is exposed to the therapeutic electromagnetic energy. This approach grossly overlooks the very critical fundamental concept that all organs and tissues are directly and indirectly systemically functionally integrated and operate with a high level of interdependence in what is essentially a Unified Vast Super System (“VAST”). The VAST includes the critical organ loop (“COL”), namely, the heart, kidneys, lungs, liver, spleen, and pancreas, gall bladder, as well as, the gastrointestinal tract (“GI Tract”), the autonomic nervous system (“ANS”), and innate immune system. Therefore, when any organ or tissue is dysfunctional or failing to any appreciable degree and/or for any appreciable length of time it is likely that the entire system (“VAST”) has been forced to compensate, and therefore, is operating in a state of homeostatic imbalance. Operating in a state of advanced and prolonged homeostatic imbalance can create unsustainable stresses and generate generalized inflammation and edema in interdependent tissues and organs which may result in secondary organ or tissue dysfunction and illness. For improved results using photo biostimulation, there exists a need for a more all-encompassing unified, integrative approach for treating a patient's VAST including those tissues and organs that have displayed symptoms of dysfunction or failure as well as the remaining functioning tissues and organs that are likely decompensating and therefore also exhibiting resultant increasing levels of inflammation and edema with the goal of restoring homeostasis and thereby support the health and functional automaticity of the body. The objective is the restoration of homeostasis beyond the isolated primarily dysfunctional organ to include the entire VAST and therefore support the healthy functional automaticity of the entire body. Numerous disease states exhibit generalized inflammation and edema. Critically and uniquely, out method applies the known anti-inflammatory effect of near infrared and infrared wavelengths, beyond the isolated dysfunctional organ, to the entire VAST, recognizing that inflammation and edema commonly emerge along the entire VAST as it decompensates.

Another area in which photo biostimulation efficiency and effectiveness may be improved is through more precise dosimetry. Perhaps because many of the biological mechanisms are not fully understood, it is currently typical practice to expose targeted organs or tissue to a wide frequency range of electromagnetic energy for varying lengths of time. However, research has shown that the biological response to electromagnetic energy tends to be wavelength dependent and biphasic. This means that there exists significant variance in biological response dependent on wavelength of the chosen electromagnetic energy, and under and overexposure of chosen effective wavelengths can compromise or eliminate the desired biological response. Therefore, to increase the effectiveness of photo biostimulation there exists a need for more precise dosimetry including both temporal exposure and wavelength control.

Further, improvements may be made to increase the effectiveness of photo biostimulation as well, such as the synergistic combination of electromagnetic and ultrasonic energy. The simultaneous use of ultrasonic and electromagnetic energy appears to assist microcirculation specifically, microvascular and microlymphatic extracellular fluid circulation, intracellular cytosol fluid circulation, and to shift the positive effects of the photo-ultrasonic biostimulation response to relatively deeper tissue (which is particularly beneficial with chronic edema, common in organ dysfunction). There exists a need for a photo biostimulation apparatus and method of use that utilizes the synergistic effects of ultrasonic energy in conjunction with electromagnetic energy.

The present disclosure distinguishes over the related art providing heretofore unknown advantages as described in the following summary.

### BRIEF SUMMARY OF THE INVENTION

The present disclosure relates to a photo-ultrasonic biostimulation device and method of use for noninvasive or minimally invasive treatment of organ dysfunction and failure by reestablishing homeostasis in the Unified Vast Super System (“VAST”) through photo-ultrasonic biostimulation. The VAST includes the critical organ loop (“COL”), namely, the heart, kidneys, lungs, liver, spleen, and pancreas, gall bladder, as well as, the gastrointestinal tract (“GI Tract”), the autonomic nervous system (“ANS”), and innate immune system. Such device and method of use is to be used concurrently with standard best medical practices of diagnosis and treatment and is designed to supplement and significantly improve outcomes.

The apparatus and method of use are designed consistent with the theory that the healthy and functional automaticity of the body is dependent on a homeostatic equilibrium that exists throughout an individual's body, is maintained by the cooperative and interdependent contributions of all cells, tissues, and organs. This homeostatic equilibrium is a dynamic balance and the net result of the body's compensatory and regulatory actions required to maintain an internal protective physiological steady state despite constantly changing external conditions. Each cell within each tissue and organ depend on this homeostatic equilibrium to continue to live and function properly, and in turn, each cell contributes toward the maintenance of homeostasis. This process is dependent upon production and consumption of existentially critical mitochondrial ATP energy molecules, which in turn is dependent upon the availability in the extracellular fluid of critical cellular inputs of O2, glucose, and molecular nutrients, molecular messengers (hormonal), and factors (enzymatic and ionic).

To restate the fundamental basis upon which the presently disclosed apparatus and method of use relies, in transitive logic:
1) Form follows function;
2) function follows homeostatic balance;
3) homeostatic balance follows:
   a) the quality, quantity, and circulation rate of the extracellular fluid which bathes all cells and is each cells' immediate external environment, and
   b) the quality, quantity, and circulation rate of the cytosol (intracellular fluid, less the nucleus and organelles);
4) These extra and intra cellular fluid properties, in turn, follow:
   a) energy available via mitochondrial ATP;
   b) microvascular inputs to the extracellular fluid of O2, glucose, molecular nutrients, molecular messengers (hormonal), and factors (ionic and enzymatic), which can be improved by mitochondrial nitric oxide and resultant microvascular dilitation; and
   c) micro lymphatic drainage of extracellular waste products and edema from the extracellular fluid, which can be improved by mitochondrial nitric oxide and ultrasonic microagitation.

The homeostatic equilibrium and the healthy functional automaticity of the body exists until one or more of an individual's internal functional systems lose their ability to contribute. Typically preceded by homeostatic disruption on the cellular and extracellular fluid level of the respective systems in decline. When this happens, the homeostatic equilibrium is disrupted causing the interdependent cells, tissues, and organs to function in a less than optimal environment (degraded pH, temperature, extracellular fluid quality, etc . . . ) and/or attempt to compensate for the failed or failing system. Both operating in a less than optimal environment and attempting to compensate for one or more failed or failing interdependent internal systems may cause remaining cells, tissues, and organs to adversely alter their function, with a resultant cascade of homeostatic imbalance through much of the Unified Vast Super System (“VAST”).

All, dysfunctional organs are in homeostatic imbalance. Characteristics include: 1) extended inflammation and edema which disrupts extracellular fluid balance and microcirculation resulting in stagnation and stasis; 2) lymphatic blockade, which degrades the quality of the extracellular fluid in which the cells bathe from physiologically supportive and protective into what is essentially the hypoxic, toxic soup of concentrated metabolic waste byproducts; and 3) micro vascular compromise, which denies the cells the minimum metabolic input requirements of oxygen, glucose, and molecular nutrients, molecular messengers (hormonal), and factors (ionic and enzymatic); 4) a decrease in the quantity, quality, and function of the mitochondria.

Further, all organ degenerative conditions, organ dysfunctions, and organ failures are characterized by dysfunctional mitochondria, specifically a reduction in mitochondrial number, size, mass, and responsiveness. At the extreme, majorities of dysfunctional mitochondria are so compromised as to be essentially inert, in a hibernating state. Organ recovery potential is based on restoring the mitochondrial profile to normal quantity, quality and function. NIR/IR photage of the mitochondria, epigenetically, via retrograde nuclear signaling, upregulates nuclear genes which code for an increase in mitochondrial biogenesis (an increase in mitochondrial number, size, mass, and responsiveness). The net effect is the restoration of cell, tissue, and organ energy state, and resultant homeostatic support.

In essence, homeostasis is critical to life and the automaticity of normal physiologic function, which is the bedrock of health. Homeostasis requires the constant production and consumption of energy, and the homeostatic imbalance occurs as cellular energy and inputs are compromised. Advanced homeostatic imbalance progressively leads to cell, tissue, and organ dysfunction. Moderate dysfunction leads to illness; extreme dysfunction leads to death.

Dysfunction resulting from homeostatic imbalance can manifest in several ways including extended inflammation and/or lymphatic blockade resulting in edema which degrades the quality of the of the extracellular fluid from which the cell both receives nutrition and expels waste. Ultrasonic microagitation addresses the compromised microvascular and microlymphatic circulation by assisting in the break up and dissolution of the edema, thereby greatly improving extracellular circulation and upgrading the quality of extracellular fluid; restoring the availability of critical cellular inputs of oxygen, glucose, and molecular nutrients, molecular messengers (hormonal), and factors (ionic and enzymatic) and reversing generalized inflammation and edema, in cell, tissue, and organs to or toward normal.

The ultrasonic energy is critical to the presently disclosed method since the more long term and chronic the inflammation, the more the associated edema changes consistency from free flowing to viscous and finally to almost gelatinous in nature. This viscosity change is almost universally overlooked by clinicians but is central to the novelty of the presently disclosed apparatus because the ultrasonic energy causes microagitation which greatly improves the break up and dissolution of viscous and/or gelatinous edema and thereby improves waste elimination in the extracellular fluid, via lymphatic/glymphatic drainage. Without, the dissolution of viscous and/or gelatinous edema a lymphatic blockade develops leading to a hypoxic, toxic stagnation and stasis of extracellular fluid causing homeostatic imbalance and cell, tissue, and organ dysfunction.

The minimum preconditions to establish or reestablish homeostasis are: 1) an adequate supply of existentially critical cellular energy or ATP; 2) adequate vascular microcirculation to supply essential inputs to the extracellular fluid; and 3) adequate lymphatic micro-drainage of cellular waste from the extracellular fluid. The presently disclosed apparatus and method of use enhances an organ's ability to meet these preconditions through a stimulated increase in production of cellular energy, enhanced vascular microcirculation and lymphatic micro-drainage through the synergistic exposure to electromagnetic and ultrasonic energy.

Research has shown that mitochondria can be stimulated with specific wavelengths of electromagnetic energy to increase its production of ATP. This is often cited as the mitochondria's primary function, however, the mitochondria also functions as a primary communication hub and is responsible for regulating cell physiology in several other important ways including controlling retrograde nuclear signaling, which epigenetically controls gene expression that effects such functions as mitochondrial biogenesis, protein synthesis and the rate of cellular mitosis. The activated mitochondria also epigenetically affects the mitochondrial genome normalizing reactive apoptosis. The photo bombarded mitochondria also increases nitric oxide availability, which critically promotes both microvascular and microlymphatic dilatation with the net result of improving toward optimal the quality of the extracellular fluid (which bathes all cells), and is the immediate external environment of all cells. This, along with increased ATP production, is absolutely essential to reestablishing homeostasis in cells, tissues, organs, and the VAST, since extracellular fluid, improved toward optimal, provides improved concentration and availability of essential cellular inputs of O2, glucose, and molecular nutrients, molecular messengers (hormonal), and factors (enzymatic and ionic).

Generally speaking, cellular functions tend to degrade or shut down significantly when a cell experiences a sizable shortage of energy. In an almost logical, triaged, physics-driven, conservation of energy, cells tend to reduce or eliminate cellular functions in the reverse order of priority and may even shut down and self-liquidate a cell entirely through reactive apoptosis should insufficient energy be available. Essentially due to severe homeostatic imbalance, the cell inputs are insufficient to maintain a minimum cellular energy state and this triggers reactive apoptosis as the body seeks to conserve and reallocate scare resources of energy and nutrients. Operating in a state of advanced and prolonged homeostatic imbalance can create unsustainable stresses on interdependent organs and tissues which may result in such cellular shutdown causing secondary organ dysfunction and illness. If reactive apoptosis cascades and accelerates, it produces a vicious cycle of cellular liquidation so extensive as to affect gross tissue dimension and precipitates organ dysfunction and failure. At the extreme this causes the phenomena of multiple organ failure “MOF” often observed in the intensive care venues.

Stimulating the mitochondria to produce additional ATP, promoting epigenetic mitochondrial biogenesis, enhancing microvascular and microlymphatic circulation such that the cell can support the body's natural healing processes and reverse dysfunction throughout the VAST and subsequently the body, is critical to restoring homeostasis and supporting the healthy functional automaticity of the body and internal health in a noninvasive manner.

The presently disclosed apparatus generates electromagnetic energy of four different effective wavelength; 628 nm, 690 nm, 812 nm, and 903 nm with a tolerance for each specified wavelength of plus or minus 5 nm. Each have been carefully selected due to their maximum biostimulating effects at progressively deeper depths of tissue penetration, ranging from superficial to organ depth. The carefully selected wavelengths may be used simultaneously, sequentially, or individually, (either continuous or pulsed), according to specific temporal protocols. The power of the electromagnetic energy in the infrared spectrum should not exceed 200 mW/cm² and the power of the electromagnetic energy in the near infrared spectrum should not exceed 40 mW/cm². This is a relatively conservative limitation compared to the 500 mW/cm² upper limitation the photo-medicine community observes for low level light therapy (LLLT). It has been noted that even shallow depth photage can have far ranging systemic effects via the circulatory, hormonal and ANS. These wavelengths have been selected to, when used in combination or individually, produce the most efficacious biostimulative effects resulting in a restoration of homeostasis at the cell, tissue and organ level. When applied in our prescribed method, to the entire VAST, we can effectively intervene, impede, arrest, and frequently reverse organ dysfunction and organ failure in a noninvasive, unified approach.

Further, the presently disclosed photo biostimulation device has been coupled with an ultrasonic energy generator. When electromagnetic energy of the above mentioned wavelengths are assisted by ultrasonic energy in the frequency range of 0.5 to 10 MHz, better results at depth are realized. The synergistic effect between the electromagnetic and ultrasonic energy increases cell, tissue, and organ response and recovery due to depth of photon penetration of tissue and improved intra and extracellular fluid circulation, due to ultrasonic microagitation (improved microcirculation and improved cell function).

While the coupling of electromagnetic and ultrasonic energy enhances the positive biostimulation effects; and the unified, integrative, noninvasive treatment approach, including exposing all organs in the VAST to enhance the bodies' ability to maintain or reestablish homeostasis; the effects of which, on all organs, appear to be biphasic, meaning that under or over exposure of wavelength that yielded positive results appear to have diminishing returns and then reverse the positive results all together. Therefore, the temporal duration of exposure and temporal interval of treatment is as important as the wavelength selection of electromagnetic energy and ultrasonic energy.

When addressing the VAST, the presently disclosed apparatus is most effective when used to in accordance with the following VAST Exposure Protocol: the heart should be exposed for two minutes; the kidneys should be exposed for one minute each; each lung should be exposed for one minute; the liver should be exposed for two minutes; the spleen and pancreas should be exposed for a combined one minute; the gastrointestinal tract and associated gall bladder should be exposed for one minute; the autonomic nervous system along the spinal tract should be exposed for one minute; and the lower extremities should be exposed for one minute each (for a total of thirteen minutes). Temporal deviation from this protocol may be necessary to accommodate differences in tissue volume and degree of edema and homeostatic imbalance in the range of plus or minus twenty-five percent.

This thirteen minute VAST Exposure Protocol should be repeated according to the VAST Schedule Protocol. For the purposes of definition the VAST Exposure Protocol repeated according to the VAST Schedule Protocol is referred to as the VAST Standard Protocol (VAST Exposure Protocol repeated according to the frequency set forth in the VAST Schedule Protocol=VAST Standard Protocol).

The Vast Schedule Protocol comprises repeating an exposure protocol (either the VAST Exposure Protocol or an adjunct exposure protocol) once a day for the first five days; once every other day up to and including the fourteenth day; and once every three days up to and including the thirtieth day; and a maintenance schedule of once or twice a week on going, as needed. “As needed,” is a determination made by the individual administering the treatment that is made based on both the apparent responsiveness and the continued existence of the symptoms or condition, or reemergence of the symptoms or degrading condition (It is not uncommon to observe regression and default without ongoing maintenance). The VAST Exposure Protocol repeated according to the VAST Schedule Protocol is referred to as the VAST Standard Protocol and is the standard method of use of the presently disclosed apparatus to restore homeostasis and health and is particularly well suited to certain specific central conditions including: 1) cardiac; 2) renal; and 3) liver dysfunction and failure.

Temporal exposure duration of the Vast Exposure Protocol and Adjunct exposure protocols are based on standard or average sized target tissues and organs. Adjustments to the temporal duration may be necessary for target tissues and organs that are not within one standard deviation of the normalized curve of recipient target tissue and organ sizes. Adjustments in the temporal duration maybe within the range of and increase or decrease of twenty-five percent.

Cardiac Failure (often termed Congestive Heart Failure) is a chronic progressive organ dysfunction in which the heart muscle is unable to pump sufficient blood through the heart to satisfy the body's needs of blood and oxygen. Associated signs and symptoms include (but are not limited to) shortness of breath, fluid retention (leg edema), and exercise intolerance. Reduced cardiac output affects cells, tissue and organs in the entire VAST. Downstream organs respond by decompensating, resulting in compromised, sub optimal function. Due to this direct and indirect systemic functional integration, the disclosed method treats the entire VAST Super System. This is a critical, unique element in our protocol.

Specifically, cardiac contractility, ejection fraction and cardiac output are compromised and arrhythmias frequently emerge, further compromising output. On a cellular and tissue level, this is due to ischemia or an injury (mechanical, chemical, thermal) which disrupts the endothelial integrity. The myocardium exhibits significant homeostatic imbalance and is characterized by prolonged inflammation, edema (often quite viscous), hypoxia, lymphatic blockade, and metabolic by product (waste) accumulation in the extracellular fluid (the immediate environment of the myocardium).

Essentially, (as in all categories of organ dysfunction and failure), there has been a shift of the quality of the extracellular fluid (the immediate environment of the myocardium) from a physiologically balanced, nurturing, steady state, capable of providing sufficient critical metabolic cellular input requirements of oxygen, glucose, molecular nutrients, molecular messengers (hormonal), and factors (ionic and enzymatic), to a stagnant, hypoxic, toxic soup.

In essence, rather than supporting the cell, this degraded stagnant, hypoxic, toxic soup poisons the cell, tissue, and organ (with cascading deleterious effects on the entire VAST). Cell inputs are insufficient to maintain minimum cellular energy state and function is degraded to a near standstill. The organ state has descended into dysfunction. At the extreme, advanced and prolonged homeostatic imbalance results in significant mitochondrial dysfunction to a near hibernating state, and triggers reactive apoptosis (self-liquidation), as the body seeks to conserve and reallocate scarce resources of energy and nutrients. If reactive apoptosis cascades and accelerates, it produces a vicious cycle of cellular liquidation so extreme as to affect gross tissue dimensions and precipitates organ failure.

The presently disclosed device and method of use is designed to specifically intervene and restore homeostasis in the extracellular fluid, as well as, increase the cell's functional energy state via increased mitochondrial ATP. This restoration of homeostatic extracellular fluid (renewing sufficient concentrations of critical cellular inputs of oxygen, glucose, molecular nutrients and molecular messengers (hormonal), and factors (ionic and enzymatic), as well as evacuating extracellular waste products, coupled with improved cellular energy state (via increased mitochondrial ATP, increased epigenetic mitochondrial biogenesis, increased rate of mitosis, and accelerated protein synthesis) promotes cellular functional recovery, increased new cellular proliferation and healing of cells, tissues and the organ (heart), as well as reverses generalized inflammation and edema. The combined effect is to support the normal automaticity of the cell, tissue and heart, with beneficial effects on all downstream organs and the entire unified VAST Super System.

The primary physiologic effects we seek to induce with the disclosure are:
(1) increased mitochondrial ATP and improved cellular energy state;
(2) increased nitric oxide availability, which promotes microvascular and micro lymphatic dilatation (thereby evacuating metabolic waste and improving delivery of critical inputs to the extracellular fluid);
(3) reduction and reversal of generalized cell, tissue, and organ inflammation and edema to or toward normal;
(4) increased rate of mitosis and enhanced protein synthesis;
(5) stimulated epigenetic mitochondrial biogenesis;
(6) normalized mitochondrial apoptosis;
(7) improved extracellular fluid and cytosol circulation via ultra sound, thereby promoting the distribution and uptake of critical cellular metabolic inputs, as well as the elimination of waste.

An added photo-ultrasonic biostimulation effect is the dissolution of viscous extracellular edema, thereby assisting the microlymphatic evacuation of large molecule metabolic extracellular waste. (Items 1, 2, 3, 4, 5 and 6 above are a direct result of photage with NIR/IR wavelengths, while item 7 is a result of exposure to ultrasonic energy).

The presently disclosed apparatus and method of use have been designed to manipulate the mitochondria, stimulate epigenetic mitochondrial biogenesis, increase the cell's functional energy state, reverse inflammation and edema, and restore homeostasis to the extracellular fluid and thereby successfully intervene, impede, arrest and frequently reverse organ dysfunction/failure. Critically, our method, by uniquely recognizing the direct and indirect functional integration of the unified, VAST Super System; directs treatment exposure along the entire VAST, producing a system wide, virtuous cycle and organ recovery, well beyond what could be achieved by treating the primary dysfunctional organ in isolation, as is common in the prevailing medical art.

Renal Dysfunction and Failure is the failure of the kidneys to adequately filter waste products from the blood. The condition may be either acute, chronic, or acute superimposed on chronic. Associated signs and symptoms (include, but are not limited to) elevated levels of serum urea, creatinine and reduced glomerular filtration rate (GFR). Typically, increased fluid causes edema in the legs, hands and face. Serum calcium, phosphate, and potassium are elevated. In later stages, urine protein is elevated there is anemia, fatigue, general health compromise, and increased risk of cardiovascular disease. Common causes are uncontrolled diabetes, uncontrolled hypertension, autoimmune disorders, polycystic kidney disease, prolonged deprived blood supply, drug overdoses, chronic overload of drugs such as antibiotics or chemotherapy. Kidney dysfunction affects cells, tissues and organs in the entire VAST. Downstream organs respond by decompensating, resulting in compromised, suboptimal function. Due to this direct and indirect systemic functional integration, our method treats the entire VAST. This is a critical, unique element in our protocol. Specifically, the glomeruli (the basic physiologic functional unit of the kidney) fails to filter. On a cellular and tissue level, this is due to ischemia or an injury (mechanical, chemical, or thermal), which disrupts the endothelial integrity. The glomeruli exhibit significant homeostatic imbalance and is characterized by prolonged inflammation, edema (often quite viscous), hypoxia, lymphatic blockade and metabolic by product (waste) accumulation in the extracellular fluid (the immediate environment of the glomeruli).

Essentially, (as in all categories of organ dysfunction and failure) there has been a shift of the quality of the extracellular fluid (the immediate environment of the glomeruli) from a physiologically balanced, nurturing, steady state, capable of providing sufficient critical metabolic cellular input requirements of oxygen, glucose, molecular nutrients, molecular messengers (hormonal), and factors (ionic and enzymatic), to a stagnant, hypoxic, toxic soup.

In essence, rather than supporting the cell, this degraded, stagnant hypoxic, toxic soup poisons the cell, tissue and organ (with cascading deleterious effects on the entire VAST Super System). Cell inputs are insufficient to maintain minimum cellular state, and function is degraded to a near standstill. The organ state has descended into dysfunction. At the extreme, advanced, prolonged homeostatic imbalance results in significant mitochondrial dysfunction to a near hibernating state and triggers reactive apoptosis, (self-liquidates) as the body seeks to conserve and reallocate scarce resources of energy and nutrients. If reactive apoptosis cascades and accelerates, it produces a vicious cycle of cellular liquidation so extensive as to affect gross tissue dimensions and precipitates organ failure.

The presently disclosed device and method of use is designed to specifically intervene and restore homeostasis in the extracellular fluid, as well as, increase the cell's functional energy state via increased mitochondrial ATP. This restoration of homeostatic extracellular fluid (renewing sufficient concentrations of critical cellular inputs of oxygen, glucose, molecular nutrients and molecular messengers (hormonal), and factors (ionic and enzymatic), as well as, evacuating extracellular waste product, coupled with improved cellular energy state (via increased mitochondrial ATP, epigenetic mitochondrial biogenesis, increased rate of mitosis and accelerated protein synthesis) promotes cellular functional recovery, increased new cellular proliferation and healing of cells, tissues and the organ (kidney) and reverses generalized inflammation and edema. The combined effect is to support the normal automaticity of the cell, tissue and kidney, with beneficial effects on all downstream organs and the entire VAST Super System.

The primary physiologic effects we seek to induce with the disclosure are:
(1) increased mitochondrial ATP and improved cellular energy state;
(2) increased nitric oxide availability, which promotes microvascular and micro lymphatic dilitation (thereby evacuating metabolic waste and improving delivery of critical inputs to the extracellular fluid);
(3) reduction and reversal of generalized cell, tissue, and organ inflammation and edema to or toward normal;
(4) increased rate of mitosis and enhanced protein synthesis;
(5) stimulated epigenetic mitochondrial biogenesis;
(6) normalized mitochondrial apoptosis;
(7) improved extracellular fluid and cytosol circulation via ultra sound, thereby promoting the distribution and uptake of critical cellular metabolic inputs, as well as the elimination of waste.
   An added photo-ultrasonic biostimulation effect is the dissolution of viscous extracellular edema, thereby assisting the microlymphatic evacuation of large molecule metabolic extracellular waste. (Items 1, 2, 3, 4, 5 and 6 above are a direct result of photage with NIR/IR wavelengths, while item 7 is a result of exposure to ultrasonic energy).

The presently disclosed apparatus and method of use have been designed to manipulate the mitochondria, stimulate epigenetic biogenesis, increase the cell's functional energy state, reverse inflammation and edema, and restore homeostasis to the extracellular fluid and thereby successfully intervene, arrest, impede, and frequestly reverse organ dysfunction/failure. Critically, our method, by uniquely recognizing the direct and indirect functional integration of the unified, VAST Super System; directs treatment exposure along the entire VAST, producing a system wide, virtuous cycle and organ recovery, well beyond what could be achieved by treating the primary dysfunctional organ in isolation, as is common in the prevailing medical art.

Liver dysfunction and failure is anything that compromises liver functions, (including, but not limited to) toxin and metabolite blood filtration, enzyme and protein production, energy storage (glycogen) and innate immune system reserve. Liver disease may be caused by viral infection, chemical toxicity or autoimmune disorders. Signs and symptoms include generalized prolonged inflammation, edema, fatty metamorphosis, fibrosis (isolated scarring) and at the extreme, cirrhosis to the point that the liver can no longer regenerate. This often leads to complications such as liver cancer. Easy bruising and prolonged bleeding, with fluid retention (edema) in the extremities and abdomen are common. Toxins may concentrate in the blood and affect concentration, memory and judgment. Insulin resistance and type II diabetes may develop. Liver dysfunction affects cells, tissues and organs in the entire VAST. Downstream organs respond by decompensating, resulting in compromised, suboptimal function. Due to this direct and indirect systemic functional integration, our method treats the entire VAST. This is a critical, unique element in our protocol. Specifically, the hepatocytes (the basic, fundamental physiologic units of the liver) fail to function, partially or completely. On a cellular and tissue level, this is due to ischemia or an injury (mechanical, chemical, or thermal), which disrupts the endothelial integrity. The hepatocytes and hepatic tissue exhibit significant homeostatic imbalance and is characterized by prolonged inflammation, edema (often quite viscous), hypoxia, lymphatic blockade, and metabolic by product (waste accumulation) in the extracellular fluid (the immediate environment of the hepatocytes).

Essentially, (as in all categories of organ dysfunction and failure) there has been a shift of the quality of the extracellular fluid (the immediate environment of the hepatocytes) from a physiologically balanced, nurturing, steady state, capable of providing sufficient critical metabolic cellular input requirements of oxygen, glucose, molecular nutrients, molecular messengers (hormonal), and factors (ionic and enzymatic), to a stagnant, hypoxic, toxic soup.

In essence, rather than supporting the cell, this degraded, stagnant, hypoxic, toxic soup poisons the cell, tissue and organ (with cascading deleterious effects on the entire VAST Super System). Cell inputs are insufficient to maintain minimum cellular state, and function is degraded to a near standstill. The organ state has descended into dysfunction. At the extreme, this results in significant mitochondrial dysfunction to a near hibernating state, advanced, prolonged homeostatic imbalance and triggers reactive apoptosis, (self-liquidates) as the body seeks to conserve and reallocate scarce resources of energy and nutrients. If reactive apoptosis cascades and accelerates, it produces a vicious cycle of cellular liquidation so extensive as to affect gross tissue dimensions and precipitates organ failure.

The presently disclosed device and method of use is designed to specifically intervene and restore homeostasis in the extracellular fluid, as well as, increase the cells functional energy state via increased mitochondrial ATP. This restoration of homeostatic extracellular fluid (renewing sufficient concentrations of critical cellular inputs of oxygen, glucose, molecular nutrients and molecular messengers (hormonal), and factors (ionic and enzymatic), as well as, evacuating extracellular waste products, coupled with improved cellular energy state (via increased mitochondrial ATP, epigenetic mitochondrial biogenesis, increased rate of mitosis and accelerated protein synthesis) promotes cellular functional recovery, increased new cellular proliferation and healing of cells, tissues and the organ (liver), and reverses generalized inflammation and edema. The combined effect is to support the normal automaticity of the cell, tissue and liver, with beneficial effects on all downstream organs and the entire VAST Super System.

The primary physiologic effects we seek to induce with the disclosure are:
(1) increased mitochondrial ATP and improved cellular energy state;
(2) increased nitric oxide availability, which promotes microvascular and micro lymphatic dilitation (thereby evacuating metabolic waste and improving delivery of critical inputs to the extracellular fluid);
(3) reduction and reversal of generalized cell, tissue, and organ inflammation and edema to or toward normal;
(4) increased rate of mitosis and enhanced protein synthesis;
(5) stimulated epigenetic mitochondrial biogenesis;
(6) normalized mitochondrial apoptosis;
(7) improved extracellular fluid and cytosol circulation via ultra sound, thereby promoting the distribution and uptake of critical cellular metabolic inputs, as well as the elimination of waste.
   An added photo-ultrasonic biostimulation effect is the dissolution of viscous extracellular edema, thereby assisting the microlymphatic evacuation of large molecule metabolic extracellular waste. (items 1, 2, 3, 4, 5, and 6 above are a direct result of photage with NIR/IR wavelengths, while item 7 is a result of exposure to ultrasonic energy).

The presently disclosed apparatus and method of use have been designed to manipulate the mitochondria, stimulate epigenetic mitochondrial biogenesis, increase the cell's functional energy state, reverse generalized inflammation and edema, and restore homeostasis to the extracellular fluid and thereby successfully intervene, arrest, impede, and frequently reverse organ dysfunction/failure. Critically, our method, by uniquely recognizing the direct and indirect functional integration of the unified, VAST Super System; directs treatment exposure along the entire VAST, producing a system wide, virtuous cycle and organ recovery, well beyond what could be achieved by treating the primary dysfunctional organ in isolation, as is common in the prevailing medical art.

The presently disclosed apparatus may also be utilized in accordance with specialized adjunct protocols tailored to specifically address additional conditions, beyond cardiac, renal, and liver dysfunction and failure. Some adjunct protocols include the steps of exposing additional tissues or organs while others are merely schedule or exposure variations of the previously disclosed VAST Standard Protocol. This disclosure sets forth adjunct protocols for the following specific health conditions: Diabetes Type I and II, bronchitis, pneumonia, pulmonary dysfunction, including chronic obstructive pulmonary disease (“COPD”), gastritis, colitis, irritable bowel syndrome (“IBS”) and Crohn's disease, multiple organ failure (“MOF”), stem cell recipient candidate, neurodegenerative conditions such as: age related cognitive and memory decline, age related depression, senility, dementia, Alzheimer's, traumatic brain injury (“TBI”), and stroke (both acute and chronic), select responsive cancers, erectile dysfunction, military and civilian immediate trauma care, and life extension/healthy aging. The common thread between these various specific health conditions and the responsiveness to photo-ultrasonic biostimualtion is the body's reaction, particularly that of the mitochondria and intracellular and extracellular fluid, to both electromagnetic and ultrasonic energy.

It is critical to note that a universal common pathophysiology underlies each and every organ dysfunction or condition previously listed (cardiac, renal, liver, diabetes type I and II, bronchitis, pneumonia, pulmonary dysfunction, chronic obstructive pulmonary disease (“COPD”), gastritis, colitis, irritable bowel syndrome (“IBS”), Crohn's disease, multiple organ failure (“MOF”), stem cell recipient candidate, neurodegenerative conditions including, age related cognitive and memory decline, age related depression, senility, dementia, Alzheimer's, traumatic brain injury (“TBI”), and stroke (both acute and chronic), select responsive cancers, erectile dysfunction, military and civilian immediate trauma care, and life extension/healthy aging). This universal, common pathophysiology, underlies and drives the physiologic dynamics which if unchecked and broadly expand, inexorably precipitate organ dysfunction and failure in each and every condition.

Due to this universal common pathophysiology, we can and have developed a universal common treatment template (with minor adjunct variations) to intervene, impede, arrest, and frequently reverse organ dysfunction and failure, in what at first glance, appear to be quite different, and unrelated conditions. In fact, they all are driven by a universal common pathophysiology and favorably respond to a common treatment template (with minor adjunct variations). The universal, common pathophysiology basis which underlies and drives all dysfunction and failure, as follows:
1) on a cellular level, ischemia or an injury (mechanical, chemical, or thermal) occurs which disrupts the endothelial integrity;
2) as the injury becomes more expansive, the basic cellular units and tissue, exhibit significant homeostatic imbalance;
3) this homeostatic imbalance is characterized by prolonged inflammation, edema (often quite viscous), hypoxia, lymphatic blockade, and metabolic (waste) accumulation in the extracellular fluid (the immediate environment of the affected cells);
4) essentially, (as in all categories of organ dysfunction and failure) there has been a shift of the quality of the extracellular fluid (the immediate environment of all cells) from a physiologically balanced, nurturing, steady state, capable of providing sufficient critical cellular requirements of oxygen, glucose, molecular nutrients, molecular messengers (hormones), and factors (ionic and enzymatic), to a stagnant, hypoxic, toxic soup. In essence, rather than supporting the cell, it poisons the cell, tissue and organ (with cascading deleterious effects on the entire VAST Super System;
5) cell inputs become insufficient to maintain minimum cellular energy state and function is degraded to a near standstill. The organ state has descended into dysfunction.
6) At the extreme, advanced and prolonged homeostatic imbalance results in the reduction in mitochondrial number, size, mass, and function which triggers reactive apoptosis (self-liquidation), as the body seeks to conserve and reallocate scarce resources of energy and resources of energy and nutrients. If reactive apoptosis cascades and accelerates, it produces a vicious cycle of cellular liquidation so extreme, as to affect gross tissue dimensions and precipitates organ failure.
7) Organ dysfunction or failure affects cells, tissues and organs in the entire VAST Super System. Downstream organs respond by decompensating, resulting in compromised, sub-optimal function.

The universal common pathophysiology, underlying and driving the physiologic dynamics of organ dysfunction and failure is responsive to the presently disclosed photo-ultrasonic biostimulation protocol. Such protocol incorporates a common protocol template (with minor adjunct variations) and has been designed to intervene, impede, arrest, and frequently reverse organ dysfunction and failure, in what, at first glance, appear to be quite different, and unrelated conditions.

The presently disclosed apparatus and method (the basis of our universal common treatment template) is designed to specifically intervene and restore homeostasis in the extracellular fluid, as well as increase the cells functional energy state via increased mitochondrial ATP, and stimulated epigenetic biogenesis.

The restoration of homeostatic extracellular fluid (renewing sufficient concentrations of critical cellular inputs of oxygen, glucose, molecular nutrients and molecular messengers (hormonal), and factors (ionic and enzymatic), as well as, evacuating extracellular waste products coupled with improved cellular energy state (via increased mitochondrial ATP, epigenetic mitochondrial biogenesis, increased rate of mitosis and accelerated protein synthesis) promotes cellular functional recovery, increased new cellular proliferation and healing of cells, tissues and the organs and reverses generalized inflammation and edema. The combined effect is to support the normal automaticity of the cell, tissue and organ, with beneficial effects on all downstream organs and the entire VAST Super System.

Universally, (applied to all organ dysfunction and failure) the primary physiologic effects we seek to induce with our device and protocols are:
(1) increased mitochondrial ATP and improved cellular energy state;
(2) increased nitric oxide availability, which promotes microvascular and microlymphatic dilatation (thereby evacuating metabolic waste and improving delivery of critical inputs to the extracellular fluid);
(3) reduction and reversal of generalized cell, tissue and organ inflammation and edema to or toward normal;
(4) increased rate of mitosis and enhanced protein synthesis;
(5) stimulated epigenetic mitochondrial biogenesis;
(6) normalized mitochondrial apoptosis;
(7) improved extracellular fluid and cytosol circulation via ultrasound, thereby promoting the distribution and uptake of critical cellular metabolic inputs, as well as, the elimination of waste. An added ultrasonic effect is the dissolution of viscous extracellular edema, thereby assisting the microlymphatic evacuation of large molecule metabolic extracellular waste (items 1, 2, 3, 4, 5 and 6) above are a direct result of photage with near infrared/infrared wavelengths, while item 7 is a result of ultrasonic exposure.

Universally, regardless of which of the many organ conditions, dysfunctions and failures treated, the presently disclosed apparatus and method of use is designed to manipulate the mitochondria, stimulate epigenetic mitochondrial biogenesis, increase cellular energy state, reverse of generalized cell, tissue and organ inflammation and edema, and restore homeostasis to the extracellular fluid; thereby, it successfully intervenes, impedes, arrests and frequently reverses organ dysfunction and failure.

Critically, the presently disclosed method, by uniquely recognizing the direct and indirect functional integration of the unified VAST Super System; directs treatment exposure along the entire VAST Super System, producing a system wide, virtuous cycle and organ recovery, well beyond what could be achieved by treating the primary dysfunctional organ in isolation, as is common in the prevailing medical art.

The organ conditions, dysfunctions, and failures listed below, all have a universal, common pathophysiology, that drives the physiologic dynamics, which if unchecked and broadly expand, inexorably precipitate these listed dysfunctions and failures. The entire list below, also favorably respond to our previously disclosed apparatus and method of use (with minor adjunct variations). The adjunct protocols are discussed in more detail in paragraphs 60 through 73.

The adjunct protocol for diabetes type I and II includes the VAST Standard Protocol plus an additional thirty seconds of exposure to the pancreas for a total of one and a half minutes of exposure each treatment. The remaining exposure schedule is the same as set forth in the VAST standard protocol.

The adjunct protocol for bronchitis, pneumonia, and pulmonary dysfunction, including chronic obstructive pulmonary disease (“COPD”), comprises the VAST Standard Protocol plus exposure of the right and left lung for an additional one minute each for a total of two minutes of exposure for each lung. The additional minute of exposure should be administered from the body surface adjacent the lungs opposing the body surface adjacent the lungs exposed during the Standard Exposure Protocol (one minute for the dorsal surface and one minute for the ventral surface of each lung). The lung exposure should be performed twice a day for the first three days, once a day for days four through ten, and once every three days up to and including the thirtieth day with a maintenance schedule of once or twice a week on going, as needed.

The adjunct protocol for gastritis, colitis, irritable bowel syndrome (“IBD”) and Crohn's disease includes the VAST Standard Protocol plus an additional one minute of exposure to the gastrointestinal tract and gall bladder (for a total of two minutes).

The adjunct protocol for multiple organ failure (“MOF”), as seen in intensive care venues, includes the VAST Exposure Protocol with an amended schedule protocol (not to be confused with the VAST Standard Protocol) of twice a day for the first five days, once a day for days six through thirty, with a maintenance schedule of once or twice a week on going, as needed.

The adjunct protocol for stem cell recipient candidates includes the VAST Exposure Protocol with an amended schedule protocol of once a day for the thirty days prior to the stem cell procedure. The purpose of such preemptive exposure is to significantly improve the prognosis of the stem cell procedure by attempting to bring a patient's recipient organ and the VAST toward optimal homeostasis prior to the stem cell procedure rather than merely injecting stem cells into a totally dysfunctional and non homeostatically balanced organ, as is common in the present methodology. Such preemptive exposure will significantly improve procedure prognosis. (Essentially, preparing the soil before the seed is planted).

An adjunct protocol also exists to address neurodegenerative conditions including: 1) age related cognitive and memory decline identified with ever increasing frequency in the global population from the sixth decade and increasing as a function of age to involve ever increasing majorities; 2) age related depression; 3) senility; 4) dementia; 5) Alzheimer's; 6) traumatic brain injury (“TBI”); and 7) stroke (both acute and chronic). Approximately three percent of infrared light has been demonstrated to penetrate through a cadaver skull and reach the hippocampus in the 800 nm range and thus such electromagnetic energy is capable of reaching and stimulating the hippocampus promoting neurogenesis and providing recovery potential. Via near infrared/infrared exposure, ATP production can be increased, existing neurons can be energized and the glymphatic removal and flushing of waste products from extra cellular fluids can be improved, as well as, improved inputs of O2, glucose, molecular nutrients, molecular messengers (hormonal), and factors (ionic and enzymatic). This applies to both the hippocampus and the cerebral cortex, and therefore, also promotes memory, attention, perceptual awareness, thought and language. The net result is both new neuron production from the hippocampus plus an improved and elevated neuron and functional brain state. (in essence providing a lifting of the “brain fog” and an improved general functional energy state, resulting in improved quantity and quality of thought production as well as memory formation, retention and recall).

The adjunct protocol for: 1) age related cognitive and memory decline identified with ever increasing frequency in global population from the sixth decade and increasing as a function of age to involve ever increasing majorities; 2) age related depression; 3) senility; 4) dementia; and 5) Alzheimer's includes the VAST Standard Protocol plus exposure to the frontal lobe for five minutes, exposure to the right temporal parietal lobe for five minutes, and exposure to the left temporal parietal lobe for five minutes. The additional exposure steps set forth are to be performed once a day for the first 10 days, once every other day for days eleven through thirty, with a maintenance schedule of once or twice a week on-going, as needed.

The adjunct protocol for: 5) traumatic brain injury (“TBI”); and 6) stroke (both acute and chronic) includes the Vast Standard Protocol plus shaving or closely cutting the hair on the patients head (the patient may grow back his or her hair in thirty days), exposing the frontal lobe for five minutes; and exposing the occipital lobe for five minutes, exposing the right temporal parietal lobe for five minutes; and exposing the left temporal parietal lobe for five minutes. The additional exposure steps should be performed pursuant to the adjunct schedule protocol of twice a day for the first three days, once a day for days four through ten, and every other day for days ten through thirty accompanied by a maintenance schedule of once or twice a week ongoing, as needed. In some embodiments of this adjunct methodology, the use of ultrasonic energy is not used as a precaution against agitative hemorrhage or because it is poorly tolerated by the patient. In such cases, the method may be performed using only electromagnetic energy; however, the beneficial biostimulation will be somewhat compromised.

Further disclosed, is an adjunct protocol tailored to address select responsive cancers. Mammals have two distinct genomes which include all of the hereditary operation instructions. One is located in each cell's nucleus, the other located in the mitochondria. In a process referred to as epigenetics, DNA modifiers that are able to turn on and off various portions of the DNA ensure DNA is appropriately expressed and sufficient levels of various proteins, enzymes, and molecular messengers are generated. Loss of proper mitochondrial function leads to nuclear and mitochondrial gene expression errors. Diseases such as select responsive cancers directly involve expression errors or loss of epigenetic control. Essentially, genes that should be switched on, are off and vice versa leading to unwanted aggressive growth.

By stimulating the mitochondria with electromagnetic energy of specific wavelengths for specifically calculated temporal durations, the presently disclosed apparatus can reestablish normal mitochondrial epigenetic apoptic regulation and normalized gene expression and the rate of nuclear cellular mitosis which, in select responsive cancers, can intervene, imped, and or arrest unwanted growth. This is accomplished by epigenetically upregulating and normalizing both nuclear and mitochondrial DNA, specifically nuclear genes that restore and normalize the rate of cellular mitosis and mitochondrial genes which restore and normalize innate protective apoptosis. Essentially, select responsive cancers are unregulated cell growth due to transcription errors and a failure of nuclear gene to control the rate of mitosis (speed limit) as well as a failure of mitochondrial genes to control innate protective apoptosis (stop sign). When functioning properly, a cell with a transcription error, or an aberrant cancer cell, will self-liquidate via self-monitoring mitochondrial protective apoptosis. Additionally, exposure of both near infrared/infrared and ultrasonic energy reverses generalized cell, tissue and organ inflammation and edema, promotes the reestablishment of extracellular fluid homeostasis, and elicits a surge in the number and activity of lymphocytes in the innate immune system, specifically, natural killer cells (“NKC”) (which seek and destroy rogue cancer cells).

The adjunct protocol specifically tailored to select responsive cancers targets the entire maturation position trail of lymphocytes including exposure to the above identified specific wavelength electromagnetic and ultrasonic energy in accordance with the following method: exposure of the long bones of the legs for one minute per leg; exposure of the thymus for one minute; exposure of the spleen for one minute; exposure of the GI secondary lymphoid tissue for one minute; exposure of the inguinal secondary lymphoid tissue for one minute; exposure of the liver for one minute; and exposure of any cancerous masses of interest for one or two minutes (depending on mass size and depth). This adjunct protocol should be performed daily for the first fourteen days and be reduced to every other day thereafter in addition to the VAST Exposure Protocol performed pursuant to a modified schedule protocol of once every three days ten to twelve hours removed from the exposure of the innate immune system.

There is an adjunct protocol for erectile dysfunction as well. This protocol includes the Vast Standard Protocol plus exposure of the penis and prostate two minutes total (one minute for the penis and one minute for the prostrate). The additional exposure steps in the adjunct method should be performed in accordance to the VAST Schedule Protocol.

There is also an adjunct protocol for military and civilian trauma care for treatment immediately after sustaining injury on the battlefield or otherwise to reduce pain, shock, and promote healing. The adjunct protocol includes the VAST Exposure Protocol plus direct exposure to the injury site for one and a half minutes. This exposure protocol should be repeated twice a day for the first three days, once a day for the fourth day through the tenth day, and once every other day for the eleventh through the thirtieth day.

Lastly, there is an adjunct protocol for life extension and healthy aging. Such protocol is recommended after identifying signs of a decline in energy and vigor, creeping fragility, frailty, or natural, age-related failure to thrive such as commonly emerges after the age of sixty. This adjunct protocol includes the VAST Standard Protocol plus exposing the frontal lobe for five minutes; exposing the right temporal parietal lobe for five minutes; and exposing the left temporal parietal lobe for five minutes. The additional steps should be performed according to an adjunct schedule protocol of once or twice a week, ongoing.

This disclosure teaches certain benefits in construction and use which give rise to the objectives described below (this applies beyond cardiac, renal, and liver failures to include all disclosed organ conditions, dysfunctions, and failures).

A primary objective is to provide biostimulation device and method of use featuring precise dosimetery and synergistic electromagnetic and ultrasonic energy to treat internal organ dysfunction and failure by assisting the reestablishment of homeostasis and thereby support the healthy functional automaticity of the body. A primary and critical insight is our recognition of the VAST, (a codependent, interconnected, unified, vast, super system of critical organs, glands, endocrine system, innate immune system, and autonomic nervous system), which must be treated integratively, not merely focusing on an apparently isolated, independent, organ dysfunction (This is due to the direct and indirect systemic functional integration of the body).

Another objective is to provide a biostimulation device and method of use featuring precise dosimetery and synergistic electromagnetic and ultrasonic energy by improving microlymphatic and microvascular circulation.

A further objective is to provide a biostimulation device and method of use featuring precise dosimetery and synergistic electromagnetic and ultrasonic energy to treat organ dysfunction and failure by increasing metabolic cellular processes through mitochondrial stimulation causing enhanced ATP production.

A still further objective is to provide a biostimulation device and method of use featuring precise dosimetery and synergistic electromagnetic and ultrasonic energy to treat organ dysfunction and failure through mitochondrial stimulation causing a reduction and reversal of generalized cell, tissue, and organ inflammation and edema throughout the entire VAST to or toward normal.

A yet still further objective is to provide a biostimulation device and method of use featuring precise dosimetery and synergistic electromagnetic and ultrasonic energy to treat organ dysfunction and failure through mitochondrial stimulation causing improved epigenetic nuclear and mitochondrial gene up regulation and improved mitochondrial retrograde nuclear signaling.

A yet still further objective is to provide a biostimulatation device and method of use featuring precise dosimetry and synergistic electromagnetic and ultrasonic energy to treat organ dysfunction and failure through mitochondrial stimulation causing improved epigenetic upregulation of nuclear genes which code for an increase in mitochondrial biogenesis (an increase in mitochondrial number, size, mass, and responsiveness), with the net result of improving cell, tissue, and organ energy state.

A yet still further objective is to provide a biostimulation device and method of use featuring precise dosimetery and synergistic electromagnetic and ultrasonic energy to treat organ dysfunction and failure through mitochondrial stimulation causing increased cell metabolic state and functions including increased protein synthesis and an improved rate of cellular mitosis via retrograde mitochondrial signaling.

A yet still further objective is to provide biostimulation device and method of use featuring precise dosimetery and synergistic electromagnetic and ultrasonic energy to treat organ dysfunction and failure through mitochondrial stimulation causing normalized mitochondrial apoptosis via mitochondrial epigenetics.

A yet still further objective is to provide biostimulation device and method of use featuring precise dosimetery and synergistic electromagnetic and ultrasonic energy to treat select responsive cancers through mitochondrial stimulation causing normalized epigenetic regulation and normalized gene expression, specifically governing normalized the rate of mitosis and normalized apoptosis, the reestablishment of extracellular fluid homeostasis, and eliciting a surge in the number and activity of lymphocytes in the innate immune system, specifically, natural killer cells.

A yet still further objective is to provide biostimulation device and method of use featuring precise dosimetery and synergistic electromagnetic and ultrasonic energy to treat various brain and neurodegenerative cognitive dysfunctions such as age related cognitive and memory decline, age related depression, senility, dementia, Alzheimer's, stoke (acute and chronic) and traumatic brain injury through mitochondrial stimulation causing improved neuron cell inputs of O2, glucose, and molecular nutrients, molecular messengers (hormonal), and factors (ionic and enzymatic) and improved circulation of the glymphatic system and its ability to flush and remove metabolic waste. The net result is both new neuron production from the hippocampus and cerebral cortex, as well as, improved and elevated neuron and functional brain energy state (in essence providing a lifting of the “brain fog” and an improved general functional energy state, resulting in improved quantity and quality of thought production as well as memory formation, retention and recall).

A yet still further objective is to provide biostimulation device and method of use featuring precise dosimetery and synergistic electromagnetic and ultrasonic energy to treat various responsive conditions such as Diabetes Type I and II, bronchitis, pneumonia, pulmonary dysfunction, including chronic obstructive pulmonary disease (“COPD”), gastritis, colitis, irritable bowel syndrome (“IBS”) and Crohn's disease, multiple organ failure (“MOF”), stem cell recipient candidates, neurodegenerative conditions such as: age related cognitive and memory decline, age related depression, senility, dementia, Alzheimer's, traumatic brain injury (“TBI”), and stroke (both acute and chronic), select responsive cancers, erectile dysfunction, military and civilian immediate trauma care, and life extension/healthy aging.

Other features and advantages of the present invention will become apparent from the following more detailed description, taken in conjunction with the accompanying drawings, which illustrate, by way of example, the principles of the presently described apparatus.

### BRIEF DESCRIPTION OF THE DRAWING(S)

The accompanying drawings are diagrams that illustrate various exemplary implementations and are part of the specification. The illustrated implementations are proffered for purposes of example, not for purposes of limitation. Illustrated elements and steps will be designated by numbers. Once designated, an element or step will be identified by the identical number throughout. Illustrated in the accompanying diagram drawings is at least one of the best mode embodiments of the present disclosure. In such drawings:

FIG. 1 is a top perspective view of an exemplary embodiment of the presently disclosed apparatus;

FIG. 2 is a bottom perspective view of an exemplary embodiment of the presently disclosed apparatus;

FIG. 3 is a block diagram featuring the various elements of an exemplary embodiment of the presently disclosed apparatus.

### DETAILED DESCRIPTION OF DRAWINGS OF AN EXEMPLARY EMBODIMENT

The above described drawings illustrate an exemplary embodiment of an apparatus in at least one of its preferred, best mode embodiments, which is further defined in detail in the following description. Those having ordinary skill in the art may be able to make alterations and modifications to what is described herein without departing from the spirit and scope of the disclosure. Therefore, it must be understood that what is illustrated is set forth only for the purposes of example, and that it should not be taken as a limitation of the scope of the present apparatus or its method of use.

Described now in detail is a noninvasive or minimally invasive biostimulation device designed to treat (intervene, impede, arrest, and frequently reverse) organ dysfunction and organ failure by reestablishing internal homeostasis both in affected organs and throughout the body using exposure to specific wavelengths of electromagnetic energy and ultrasonic energy for specific temporal durations.

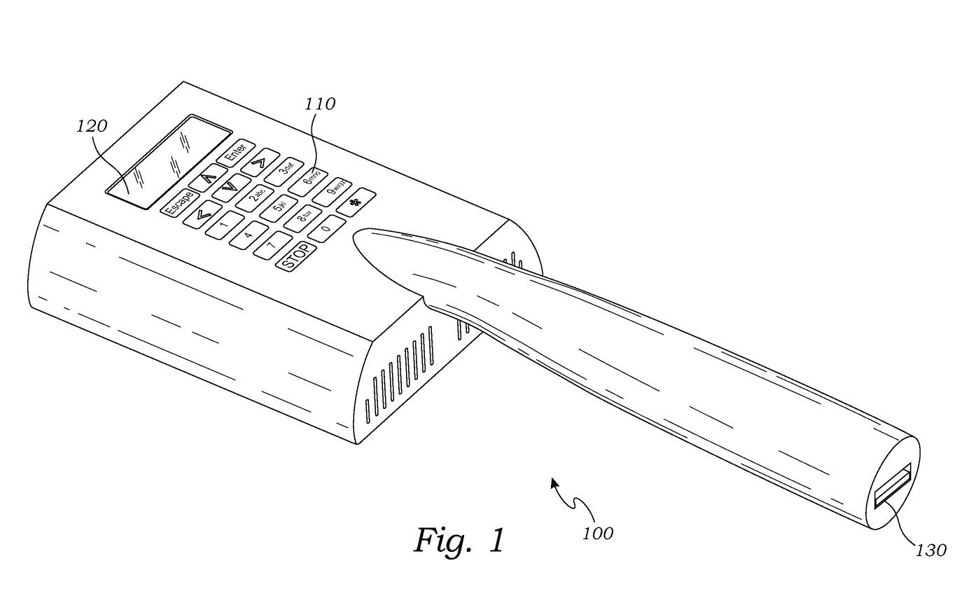

FIG. 1 is an illustration of an exemplary hand-held embodiment of the presently disclosed biostimulation device 100. The hand-held embodiment should not be viewed as limiting but rather as one of many possible embodiments. Other embodiments may include devices which are miniature implantable, small scale wearable, hand held, or large scale such as seats, stand-up tubes, or clam shell beds. Further embodiments may include both large and small scale veterinarian configurations for both large and small animals.

The illustrated exemplar device 100 features a input component in the form of an alphanumerical keypad 110 including directional navigation keys by which an operator may enter instructions or scroll through and choose among a menu of instructional options. An alphanumerical keypad 110 is one of many possible input components 110. Other embodiments may feature different input component technologies such as a touchscreen display or integration capabilities such that the apparatus may integrate with a separate computer featuring an input component, either wirelessly or through a physical interface. The important feature of the input component is the ability to interface with the human operator in a manner that allows the operator to provide the apparatus with instructions. FIG. 1 also illustrates an output component in the form of a display screen 120 through which the apparatus 100 may interface with the human operator. Similarly, other embodiments may feature output components that manifest in many forms as well so long as the component can interface with the human operator and output informational communication in a meaningful way.

FIG. 1 also illustrates a port 130 located on the distal end of the exemplar embodiment. This embodiment is included for purposes of illustration and is not meant to be limiting. Such a port 130 may provide access to power required to generate both the electromagnetic and ultrasonic energy and/or may also facilitate input and output of data such as instruction and performance metrics from an external source. Other embodiments may include an alternative power source arrangement such as a battery pack or permanent power cord.

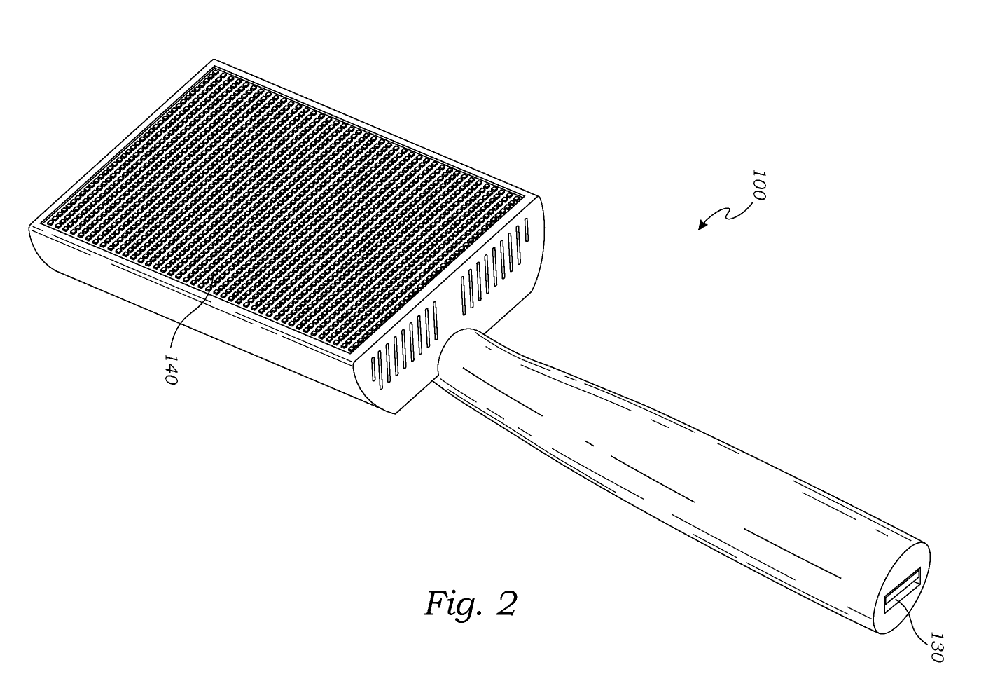

FIG. 2 illustrates a bottom prospective view of an exemplary embodiment of the presently disclosed apparatus 100. The bottom surface is configured to generate electromagnetic energy of four specific wavelengths defined within a tolerance of plus or minus 5 nm. In the embodiment illustrated in FIG. 2 this is achieved through an array of light emitting chips 140 mounted directly to the chassis of the exemplary embodiment in a chip-on-device orientation. This embodiment is not meant to be limiting. In some embodiments the chips 140 may be identical in nature merely providing a sufficient capacity through numbers, while in other embodiments the chips 140 may be comprised of a variety of different material constructions specialized to generate electromagnetic energy of specific wavelengths. The use of multiple chips 140 allows for a more evenly distributed electromagnetic fluence and greater electro-thermal efficiency, however a singular or small number of electromagnetic-energy generators 180 may be utilized as well.

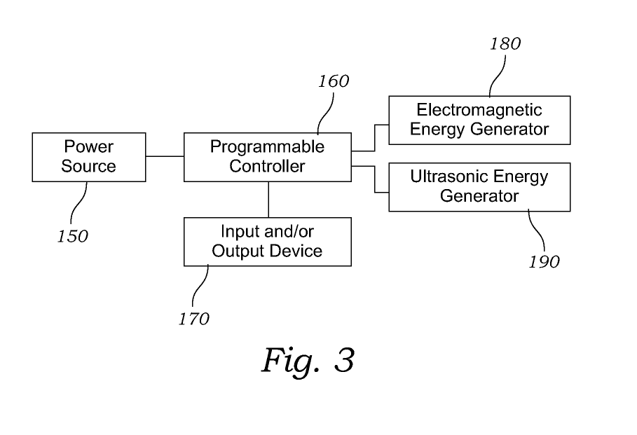

FIG. 3 is a block diagram of the basic functional components of the presently disclosed apparatus. The presently disclosed apparatus includes both an electromagnetic energy generator 180 and an ultrasonic energy generator 190 capable of generating electromagnetic and ultrasonic energy of the specifically identified wavelengths. Regulating the electromagnetic and ultrasonic energy dosimetry including exposure intensity and duration is a controller 160. In many embodiments, the controller 160 is programmable allowing the operator to alter or fine tune the dosimetry to accommodate particular preferences or to accommodate patient size, either large or small.

FIG. 3 reiterates that the presently disclosed exemplar apparatus includes both an input and output device 170. There is a broad range in the level of sophistication that may be incorporated. Some embodiments may be as simple as a start button and a light or tone indicating that the predetermined exposure duration has commenced and/or has expired. Other embodiments may include detailed information regarding the dosimetry, frequency, duration (pulsed or continuous), power and cumulative energy delivered (electromagnetic and ultrasonic). This information may vary for the various embodiments such as small and large scale formats, ranging from miniature implantable, to small wearable, handheld, and large scale such as chairs, stand-up tubes, and clam shell beds and embodiments configured for use in veterinarian applications, both small and large scale.

The enablements described in detail above are considered novel over the prior art of record and are considered to be critical to the operation of at least one aspect of the apparatus and its method of use, and to the achievement of the above-described objectives. The words used in this specification to describe the instant embodiments are to be understood not only in the sense of their commonly defined meanings, but to include by special definition in this specification: structure, material, or acts beyond the scope of the commonly defined meanings. Thus, if an element can be understood in the context of this specification as including more than one meaning, then its use must be understood as being generic to all possible meanings supported by the specification and by the word(s) describing the element.

The definitions of the words or drawing elements described herein are meant to include not only the combination of elements which are literally set forth, but all equivalent structures, materials or acts for performing substantially the same function in substantially the same way to obtain substantially the same result. In this sense it is therefore contemplated that an equivalent substitution of two or more elements may be made for any one of the elements described and its various embodiments or that a single element may be substituted for two or more elements in a claim.

Changes from the claimed subject matter as viewed by a person with ordinary skill in the art, now known or later devised, are expressly contemplated as being equivalents within the scope intended and its various embodiments. Therefore, substitutions, now or later known to one with ordinary skill in the art, are defined to be within the scope of the defined elements. This disclosure is thus meant to be understood to include what is specifically illustrated and described above, what is conceptually equivalent, what can be obviously substituted, and also what incorporates the essential ideas.

The scope of this description is to be interpreted only in conjunction with the appended claims and it is made clear, here, that the named inventors believe that the claimed subject matter is what is intended to be patented.

What is claimed is:

1. An apparatus for noninvasive biostimulation, said apparatus comprising:
a controller;
an electromagnetic energy generator; and
an ultrasonic energy generator, wherein said controller is in communication with both said electromagnetic energy generator and said ultrasonic energy generator and is capable of controlling respective generation intensity and duration.

2. An apparatus of claim 1 further comprising:
at least one input device in communication with said controller wherein said input device is capable of receiving external instructions directed by an operator and relating said external instructions to said controller.

3. An Apparatus of claim 2 further comprising:
at least one output device in communication with said controller capable of communicating ultrasonic and electromagnetic energy generation data to an operator.

4. An Apparatus of claim 3 wherein said controller is a programmable controller capable of receiving, storing, and executing operator generated programming.

5. An Apparatus of claim 4 where said electromagnetic generator generates electromagnetic energy of the wavelengths 628 nm, 690 nm, 812 nm, and 903 nm within a tolerance for each specified wavelength of plus or minus 5 nm.

6. An Apparatus of claim 5 wherein said ultrasonic energy generator generates ultrasonic energy of the frequency range of 0.5 to 10 MHz.

7. An Apparatus of claim 4 wherein said programmable controller is capable of selecting the wavelength and duration of the energy generated by said electromagnetic energy generator and said ultrasonic energy generator.

8. An apparatus of claim 5 wherein said electromagnetic energy is generated by light emitting chips mounted directly to the external chassis of the apparatus.

9. A Vast Exposure Protocol for treating cardiac, renal, and liver dysfunction and/or failure via photo-ultrasonic biostimulation, said VAST Exposure Protocol comprising the steps of:
a. exposing the heart to targeted electromagnetic energy and ultrasonic energy of predetermined wavelengths for a period of two minutes;
b. exposing the kidneys to targeted electromagnetic energy and ultrasonic energy of predetermined wavelengths for a period of one minute each;
c. exposing the lungs to targeted electromagnetic energy and ultrasonic energy of predetermined wavelengths for a period of one minute each;
d. exposing the liver to targeted electromagnetic energy and ultrasonic energy of predetermined wavelengths for a period of two minutes;
e. exposing the spleen and pancreas to targeted electromagnetic energy and ultrasonic energy of predetermined wavelengths for a combined period of one minute;
f. exposing the gastrointestinal tract and associated gall bladder to targeted electromagnetic energy and ultrasonic energy of predetermined wavelengths for a period of one minute;
g. exposing the autonomic nervous system along the spinal tract to targeted electromagnetic energy and ultrasonic energy of a predetermined wavelengths for a period of one minute;
h. exposing the lower extremities to targeted electromagnetic energy and ultrasonic energy of a predetermined wavelengths for a period of one minute each;

10. The method of claim 9 repeated according to the VAST Schedule Protocol defining the VAST Standard Protocol, said VAST Schedule Protocol comprising the steps of:
repeating the steps set forth in claim 9 once a day for the first five days of treatment;
repeating the steps set forth in claim 9 once every other day for the sixth through fourteenth day of treatment;
repeating the steps set forth in claim 9 once every three days from the fifteenth through thirtieth day of treatment;
repeating the steps set forth in claim 9 thereafter once or twice a week, on an ongoing, as needed basis.

11. The method of claim 10 wherein the predetermined wavelengths of electromagnetic energy are 628 nm, 690 nm, 812 nm, and 903 nm within a tolerance for each specified wavelength of plus or minus 5 nm and the predetermined frequency range of ultrasonic energy of 0.5 to 10 MHz.

12. The method of claim 11 wherein the predetermined temporal exposure period for both electromagnetic energy and ultrasonic energy is increased twenty-five percent for recipients whose target organs or tissues are more than one standard deviation larger than the average recipient's target organs or tissue size and decreased twenty-five percent for recipients whose target organs or tissues are more than one standard deviation smaller than the average recipient's target organs or tissue size.

13. An adjunct method of treating select responsive cancer via photo-ultrasonic biostimulation comprising the method of claim 12 further including the steps of:
a. exposing the long bones of the legs to targeted electromagnetic energy of the wavelengths 628 nm, 690 nm, 812 nm, and 903 nm within a tolerance for each specified wavelength of plus or minus 5 nm and ultrasonic energy having a frequency range of 0.5 to 10 MHz for a period of one minute per leg;
b. exposing the thymus to targeted electromagnetic energy of the wavelengths 628 nm, 690 nm, 812 nm, and 903 nm within a tolerance for each specified wavelength of plus or minus of 5 nm and ultrasonic energy having a frequency range of 0.5 to 10 MHz for a period of one minute;
c. exposing the spleen to targeted electromagnetic energy of the wavelengths 628 nm, 690 nm, 812 nm, and 903 nm within a tolerance for each specified wavelength of plus or minus 5 nm and ultrasonic energy having a frequency range of 0.5 to 10 MHz for a period of one minute;
d. exposing the GI secondary lymphoid tissue to targeted electromagnetic energy of the wavelengths 628 nm, 690 nm, 812 nm, and 903 nm within a tolerance for each specified wavelength of plus or minus 5 nm and ultrasonic energy having a frequency range of 0.5 to 10 MHz for a period of one minute;
e. exposing the inguinal secondary lymphoid tissue to targeted electromagnetic energy of the wavelengths 628 nm, 690 nm, 812 nm, and 903 nm within a tolerance for each specified wavelength of plus or minus 5 nm and ultrasonic energy having a frequency range of 0.5 to 10 MHz for a period of one minute;
f. exposing the liver to targeted electromagnetic energy of the wavelengths 628 nm, 690 nm, 812 nm, and 903 nm within a tolerance for each specified wavelength of plus or minus 5 nm and ultrasonic energy having a frequency range of 0.5 to 10 MHz for a period of one minute;
g. exposing any cancerous masses of interest to targeted electromagnetic energy of the wavelengths 628 nm, 690 nm, 812 nm, and 903 nm within a tolerance for each specified wavelength of plus or minus 5 nm and ultrasonic energy having a frequency range of 0.5 to 10 MHz for a temporal period within the range of one minute to two minutes.
h. repeating the steps a through g, once a day for the first fourteen days;
i. repeating the steps a through g once every other day from the fourteenth days to the thirtieth day, and on-going as needed;
j. repeating the steps a through h once or twice a week within twelve hours of performing steps of claim 12, on-going, as needed.

14. An adjunct method for treating diabetes type I and II via photo-ultrasonic biostimulation comprising the method of claim 12 wherein step e of claim 9 consists of the pancreas being exposed to targeted electromagnetic energy for an additional period of one half minute for a total of one and one half minutes.

15. An adjunct method of treating bronchitis, pneumonia and pulmonary dysfunction, including chronic obstructive pulmonary disease (“COPD”) via photo-ultrasonic biostimulation comprising the method of claim 12 further comprising the steps of:
a. exposing the right and left lung to targeted electromagnetic energy of the wavelengths 628 nm, 690 nm, 812 nm, and 903 nm within a tolerance for each specified wavelength of plus or minus 5 nm and ultrasonic energy having a frequency range of 0.5 to 10 MHz for an additional period of one minute each for a total of two minutes each;
b. repeating step a twice a day for the first three days;
c. repeating step a once a day for day fourth through day ten;
d. repeating step a once every three days for day eleven through day thirty; and
e. repeating step a once or twice a week ongoing as needed.

16. An adjunct method of treating, gastritis, colitis, irritable bowel syndrome and Crohn's disease via photo-ultrasonic biostimulation comprising the method of claim 12 wherein step f of claim 9 consist of:
exposing gastrointestinal tract and the associated gall bladder to targeted electromagnetic energy and ultrasonic energy of predetermined wavelengths for an additional one minute for a total of two minutes.

17. An adjunct method of treating multiple organ failure via photo-ultrasonic biostimulation comprising the method of claim 9 further comprising the steps of:
a. repeating the steps set forth in claim 9 twice a day for the first five days of treatment wherein the predetermined wavelengths of electromagnetic energy are 628 nm, 690 nm, 812 nm, and 903 nm within a tolerance for each specified wavelength of plus or minus 5 nm and the predetermined frequency range of ultrasonic energy of 0.5 to 10 MHz;
b. repeating the steps set forth in claim 9 once a day for the sixth through thirtieth day of treatment wherein the predetermined wavelengths of electromagnetic energy are 628 nm, 690 nm, 812 nm, and 903 nm within a tolerance for each specified wavelength of plus or minus 5 nm and the predetermined frequency range of ultrasonic energy of 0.5 to 10 MHz; and
c. repeating the steps set forth in claim 9 thereafter once or twice a week on an ongoing, as needed wherein the predetermined wavelengths of electromagnetic energy are 628 nm, 690 nm, 812 nm, and 903 nm within a tolerance for each specified wavelength of plus or minus 5 nm and the predetermined frequency range of the ultrasonic energy is 0.5 to 10 MHz.

18. An adjunct method for assisting stem cell recipients via photo-ultrasonic biostimulation comprising the method of claim 9 further comprising the steps of:
repeating the steps set forth in claim 9 once a day for the thirty days prior to the stem cell procedure wherein the predetermined wavelengths of electromagnetic energy are 628 nm, 690 nm, 812 nm, and 903 nm within a tolerance for each specified wavelength of plus or minus 5 nm and the predetermined frequency range of ultrasonic energy is 0.5 to 10 MHz.

19. An adjunct method of treating traumatic brain injury and stroke (acute and chronic) via photo biostimulation comprising the method of claim 12 further comprising the additional steps of:
a. shaving recipient's head, said recipient may regrow hair in thirty days;
b. exposing the frontal lobe to targeted electromagnetic energy of the wavelengths 628 nm, 690 nm, 812 nm, and 903 nm within a tolerance for each specified wavelength of plus or minus 5 nm and ultrasonic energy having a frequency range of 0.5 to 10 MHz for five minutes;
c. exposing the right temporal parietal lobe and hippocampus to targeted electromagnetic energy of the wavelengths 628 nm, 690 nm, 812 nm, and 903 nm within a tolerance for each specified wavelength of plus or minus 5 nm and ultrasonic energy having a frequency range of 0.5 to 10 MHz for five minutes;
d. exposing the left temporal parietal lobe and hippocampus to targeted electromagnetic energy of the wavelengths 628 nm, 690 nm, 812 nm, and 903 nm within a tolerance for each specified wavelength of plus or minus 5 nm and ultrasonic energy having a frequency range of 0.5 to 10 MHz for five minutes;
e. exposing the occipital lobe to targeted electromagnetic energy of the wavelengths 628 nm, 690 nm, 812 nm, and 903 nm within a tolerance for each specified wavelength of plus or minus 5 nm and ultrasonic energy having a frequency range of 0.5 to 10 MHz for five minutes;
f. repeating additional steps a through e once a day for the first ten days;
g. repeating additional steps a through e every other day for days eleven through thirty;
h. repeating additional steps b through e once or twice a week on going, as needed.

20. An adjunct method of treating neurodegenerative and age related cognitive memory decline, age related depression, senility, dementia, and Alzheimer's via via photo-ultrasonic biostimulation comprising the method of claim 12 further comprising the additional steps of:
a. exposing the frontal lobe to targeted electromagnetic energy of the wavelengths 628 nm, 690 nm, 812 nm, and 903 nm within a tolerance for each specified wavelength of plus or minus 5 nm and ultrasonic energy having a frequency range of 0.5 to 10 MHz for five minutes;
b. exposing the right temporal parietal lobe and hippocampus to targeted electromagnetic energy of the wavelengths 628 nm, 690 nm, 812 nm, and 903 nm within a tolerance for each specified wavelength of plus or minus 5 nm and ultrasonic energy having a frequency range of 0.5 to 10 MHz for five minutes;
c. exposing the left temporal parietal lobe and hippocampus to targeted electromagnetic energy of the wavelengths 628 nm, 690 nm, 812 nm, and 903 nm within a tolerance for each specified wavelength of plus or minus 5 nm and ultrasonic energy having a frequency range of 0.5 to 10 MHz for five minutes;
d. repeating additional steps a through c twice a day for the days one through three;
e. repeating additional steps a through c once a day for the days four through ten;
f. repeating additional steps a through c every other day for days eleven through thirty;
g. repeating additional steps a through c once or twice a week on going, as needed basis.

21. An adjunction protocol for erectile dysfunction via photo-ultrasonic biostimulation comprising the method of claim 12 further comprising the steps of:
i. exposing both the penis and prostate to targeted electromagnetic energy of the wavelengths 628 nm, 690 nm, 812 nm, and 903 nm within a tolerance for each specified wavelength of plus or minus 5 nm and ultrasonic energy having a frequency range of 0.5 to 10 MHz for one minute each.

22. An adjunct protocol for military and civilian trauma care via photo-ultrasonic biostimulation, said method comprising method of claim 12 further comprising the additional step of:
a. exposing the injured tissue to targeted electromagnetic energy of the wavelengths 628 nm, 690 nm, 812 nm, and 903 nm within a tolerance for each specified wavelength of plus or minus 5 nm and ultrasonic energy having a frequency range of 0.5 to 10 MHz for one and a half minute; and
b. repeating said additional step twice a day for the first three days, once a day for days four through ten, and once every other day for days eleven through thirty.

23. An adjunct protocol for life extension and healthy aging via photo-ultrasonic biostimulation, said method comprising the method of claim 12 further comprising the additional steps of:
a. exposing the frontal lobe to targeted electromagnetic energy of the wavelengths 628 nm, 690 nm, 812 nm, and 903 nm within a tolerance for each specified wavelength of plus or minus 5 nm and ultrasonic energy having a frequency range of 0.5 to 10 MHz for five minutes;
b. exposing the right temporal parietal lobe and hippocampus to targeted electromagnetic energy of the wavelengths 628 nm, 690 nm, 812 nm, and 903 nm within a tolerance for each specified wavelength of plus or minus 5 nm and ultrasonic energy having a frequency range of 0.5 to 10 MHz for five minutes;
c. exposing the left temporal parietal lobe and hippocampus to targeted electromagnetic energy of the wavelengths 628 nm, 690 nm, 812 nm, and 903 nm within a tolerance for each specified wavelength of plus or minus 5 nm and ultrasonic energy having a frequency range of 0.5 to 10 MHz for five minutes;
d. repeating said additional steps once or twice a week, ongoing.
